(19) United States
(12) Patent Application Publication
(10) Pub. No.: US 2018/0036359 A1
(43) Pub. Date: Feb. 8, 2018

(54) PROCESS TO OBTAIN A NUTRACEUTICAL FORMULATION OF AMARANTH AND ITS USE IN TREATING HUMAN BEHAVIOR DISORDERS

(71) Applicant: Manuel Soriano Garcia, Benito Juarez (MX)

(72) Inventor: Manuel Soriano Garcia, Benito Juarez (MX)

(21) Appl. No.: 15/120,540

(22) PCT Filed: Apr. 12, 2016
(86) PCT No.: PCT/MX2016/000035

§ 371 Date: Aug. 22, 2016

(30) Foreign Application Priority Data
Apr. 30, 2015 (MX) MX/A/2015/005574

(51) Int. Cl. A61K 36/21

(52) U.S. Cl. CPC A61K 36/21; A61K 2236/13; A61K 2236/33; A61K 2236/50

(57) ABSTRACT

The present invention relates to a novel process to manufacture a nutraceutical formulation of amaranth, made from amaranth whole seeds. The nutraceutical formulation of amaranth obtained by the present invention has unique characteristics for addressing problems of great relevance in the priority field of public health, which significantly affects people in general, that is to say, the use in treating human behavioral disorders such as depression, stress, anxiety, and extreme cases like schizophrenia and bipolar disorder

### FIELD OF THE INVENTION

The present invention pertains to the food and nutraceutical industry, and relates directly to the method to obtain a nutraceutical formulation of amaranth having therapeutic properties, from amaranth seeds, Amaranthus hypochondriacus.

The invention relates to a novel nutraceutical formulation with different active nutrients, said formulation comprising proteins having a high contents of essential amino acids, non-saturated fatty acids, squalene, antioxidants and carbohydrates for its use in treating behavioral disorders such as depression, stress and anxiety (DSA), schizophrenia and bipolar disorder.

### BACKGROUND OF THE INVENTION

Amaranth belongs scientifically to the dicotyledon class, order Centrospermae and the species and genus Amaranthus L. (Amaranthaceae family), it is widely spread around the world, particularly in tropical, sub-tropical and temperate climate regions.

In the Americas only three of them are cultivated: Amaranthus hypochondriacus, indigenous to Mexico; A. cruentus, indigenous to Guatemala Southeast Mexico; and A. caudatus, indigenous to South America.

Amaranth has a great nutritional value, due to the variety of the forms in which it is consumed: as grains, as vegetable or as fodder, although it is more commonly known in its puffed cereal form called ‘alegria’ in Mexico. Amaranth leaves contain 3.5 g of protein compared with 3.2 g in spinaches, and 2.9 g in Swiss chard, which were introduced to Mexico. Moreover, the Amaranth still has more significance due to its calcium content, which can be as much as about 267 milligrams, more than that of spinach which is of about 93 milligrams. Furthermore, Amaranth contains phosphorus, iron, retinol (vitamin A), and niacin (1.4 milligrams vs. 0.5 milligrams in chard).

The embryo of this plant is large, constituting a good source of lipids and proteins (storage proteins). Storage proteins have a high expression and accumulation level in grains, which plays a significant role in human nutrition.

The total protein content present in amaranth grains (13.2-18.4%) are in the middle among cereals (8.5-14.0%), and legumes (25-35%). For example, protein content in other cereals is: 10-12.8% in wheat, 9-9.5% in corn, 5.6-6.5% in rice, 13-15% in oats, 11-14% in rye, and 35-40% in soybean (USDA & National Research Council). Also, this protein has a better amino acid balance compared with legumes and cereals.

Several studies about processes to obtain amaranth protein concentrates and isolates have been published; and also about obtaining different protein fractions and the effect of conditions used in the preparation on functional, physiological and technological properties of isolates. All of these studies have investigated protein concentration at laboratory level, while the study to obtain concentrates at pilot level is poor or inexistent. This stage is key as a middle scale step in order to eventually establish the foundations of industrial process know-how.

The traditional alkali extraction and isoelectric precipitation methodology, is the chosen one for many authors to obtain grain protein isolates. Also, the effect of extraction conditions and thermal stability of grain protein isolates have been evaluated. The composition of the main grain protein fractions and the denaturing grade of protein isolates depend on and can be controlled by selecting different pH combinations for the extraction and precipitation operations.

Alternatively, three alternative processes to obtain vegetable protein preparations have been tested: 1) the use of semipermeable reverse osmosis membranes; ultrafiltration equipment; and microfiltration, which allows for water removal; water and small organic molecules; and water, small organic molecules, and proteins, respectively, at pilot plant or industrial scale, and particularly for oleaginous protein concentration and purification; 2) the use of dialysis as a process for vegetable protein concentration; 3) the use of micellization to produce vegetable protein isolates with higher protein yield. Mild treatments in micellization imply a product with less protein denaturing and improved functional properties.

Human behavioral disorders such as depression, stress and anxiety (DSA), schizophrenia and early or preliminary bipolar disorder, can be described as the condition of a person, which feels sad, melancholic, unhappy, dejected or defeated. Most people feel like this from time to time for short periods. However, clinical depression is a mood disorder, where sadness, loss, anger or frustration feelings interfere with daily life for a long period of time.

Some symptoms of DSA, schizophrenia and bipolar disorder include: Irritable or low mood, most of the time; Loss of pleasure in common activities and sexuality; Difficulty in falling asleep or oversleeping; Great change in appetite, often with weight gain or loss; Tiredness and lack of energy; Uselessness, lack of self-esteem, and culpability feelings; Concentration difficulties; Fast or slow movements; Inactivity and withdrawal from usual activities; Pathological sadness and abandonment feelings; Repetitive death or suicidal thoughts leading to its perpetration; Memory decline and frequent distractions; Anguish and anxiety crisis; Decrease in working performance; Delirium, hallucinations, flat affect; Disorganized speech and conduct; and Sudden changes in mood.

Schizophrenia is a disorder characterized by a wide spectrum of cognitive and emotional impairments including delirium and hallucinations involving all senses, speech, and disorganized behavior, as well as inappropriate emotions, which are considered as bad interpretation of reality and delirious disorders, among others.

Bipolar disorder appears as an abnormal and persistently high, expansive or irritable state of mind lasting at least for one week. Persons can exhibit exaggerated self-esteem, have a decreased need for sleep, they can be more talkative than normal, have drain of ideas, psychomotor agitation and excessive implication in pleasuring activities, among others.

Whatever the main depression, stress, anxiety, schizophrenia, and bipolar disorder symptoms are in their early or preliminary stage, these are an advisory signal indicating the necessity to make important changes in lifestyle and/or in oneself, and one must realize which changes are relevant so as to put into effect, as soon as possible, an action plan to overcome this serious situation and start the change to a more satisfactory life.

There are two United States Patents reported:

(1) U.S. Pat. No. 4,911,943 to Slimak Karen, 1990, titled “Process for products from amaranth”, and (2) U.S. Pat. No. 5,789,012 to Slimak Kara, 1998, titled “Products from sweet potatoes, cassava, edible aroids, amaranth, yam, lotus, potatoes and other roots, seeds and fruits”.

The referred patents describe the production of various flours and related amaranth products, which are for daily consumption (bread, pancakes, rolls, imitation corn bread, amaranth wafers, bagguettes, cookies, doughnuts, pretzels, crepes, tortillas, chips, amaranth imitation mayonnaise, amaranth milk, pasta and pudding) and industrial products (prepared to make pancakes, cake dough, and cooked amaranth flour).

In example 19 of U.S. Pat. No. 4,911,943, and example 125 of U.S. Pat. No. 5,789,012 a preparation of amaranth milk from amaranth flour is disclosed, and only describe a process, wherein flour is mixed with water, without considering the quality and palatability of the obtained product, nor the nutritional properties. It is worth mentioning that the production method of the amaranth product of the present invention from amaranth as raw material is totally different from the procedure described in the referred US Patents.

There are two Mexican Patents and one Patent Application by the present inventor and are the following:

Mexican Patent MX278682 to Soriano Garcia de 2010, from Universidad Nacional Autónoma de México, titled “Producto de amaranto nutritivo, su proceso de elaboración y su use en la alimentación de individuos sanos y/o pacientes con algún tipo de desorden metabólico” (Nutritious amaranth product, its manufacturing process and its use in feeding healthy individuals and/or patients suffering from a metabolic disorder), discloses the production of an amaranth product obtained from puffed and defatted amaranth.

Then in a second invention to Soriano Garcia, a new process to prepare a liquid nutritious beverage from amaranth described in MX287913 Patent granted in 2011 titled “Nuevo proceso de elaboración y estabilización de un producto nutritivo de amaranto” (New process for manufacturing and stabilizing a nutritious product from amaranth), where reference is made to the preparation of a liquid amaranth product from whole amaranth seeds as raw material.

In a third invention to Soriano Garcia, a process to prepare a nutritious beverage from powdered amaranth, as well as its use in addressing the malnutrition problem, diabetes and obesity in Mexico is disclosed. The present invention is currently embodied in the Patent Application MX/A/2013/002550 filed on Mar. 5, 2013, titled “Desarrollo tecnológico de una bebida nutritiva de amaranto en polvo, así como su use en la desnutrición, diabetes y obesidad” (Technological development of a nutritious beverage made from powdered amaranth, and its use in malnutrition, diabetes and obesity). This application describes the use of a powdered amaranth product to be prepared in liquid form and fed to healthy individuals, as well as people with any metabolic disorder, such as lactose and cow milk casein intolerance, alimentary allergies, and overweight.

In the present invention, a novel process to obtain a nutraceutical formulation of amaranth having therapeutic properties is disclosed, wherein said process differs substantially from the procedures described in the above patents, in that it uses different methodologies to obtain a powdered product with high quality nutrients.

It is worth mentioning that to date there is no prior art describing any information that anticipates to or disclose a nutraceutical formulation of amaranth for use in treating depression, stress, anxiety (DEA), schizophrenia or bipolar disorder.

The present invention describes a technological development to obtain a nutraceutical formulation of amaranth having therapeutic properties, and to serve as a viable alternative in public health issues regarding behavioral disorders such as depression, stress, anxiety (DSA), schizophrenia and bipolar disorder.

### OBJECTS OF THE INVENTION

The main object of the present invention is to develop a nutraceutical food formulation having therapeutic properties for human behavioral disorders such as depression, stress, anxiety (DSA), schizophrenia and bipolar disorder.

Another object of the invention relates to the development of a novel nutraceutical formulation, characterized in that it contains active nutrients, comprising soluble proteins having a high content of essential amino acids, non-saturated fatty acids, squalene, antioxidants and carbohydrates as active ingredients, which are obtained from amaranth seeds, Amaranthus hypochondriacus.

Another object of the invention is the use of a powdered form, which provides the product a higher chemical and bacteriological stability.

Another object of the present invention is to provide a longer shelf life. The powdered product does not require continuous refrigeration; its handling is simple and has a shelf life that makes it useful to the user without causing any harm to its health and/or without losing its functionality.

It is worth mentioning that the present invention is novel in that there is no other product in the market having a nutraceutical formulation of amaranth with therapeutic properties, and used as a viable alternative in public health issues regarding behavioral disorders, such as depression, stress, anxiety (DSA), schizophrenia, and bipolar disorder.

Other objects and aspects of the present invention will be obvious to those with ordinary skills when considering the present disclosure.

### DETAILED DESCRIPTION OF THE INVENTION

The present invention relates to a novel nutraceutical formulation and its use in treating behavioral disorders such as depression, stress, anxiety (DSA), schizophrenia, and bipolar disorder, mainly characterized by its diverse active nutrients, comprising soluble proteins having a high content of essential amino acids, non-saturated fatty acids, squalene, antioxidants and carbohydrates as active ingredients, which are obtained from amaranth seeds Amaranthus hypochondriacus. All the active ingredients allow for the production of a product having a suitable powdered nutraceutical formulation, of interest in treating DSA, schizophrenia, bipolar disorder, but that can also be used by healthy individuals.

In the present specification the term “nutraceutical” is derived from nutrition and pharmaceutical, and defines “food having a beneficial effect specifically in health”, and refer to “foodstuff natural or processed that contain ingredients that perform a specific activity on physiological functions of the human body, improving physical capacity and mental state. The main functions are related with an optimal growth and development, the normal activity of the cardiovascular system, the prevention of cardiovascular, liver and degenerative diseases such as cancer, providing antioxidants and protecting the gastrointestinal system, the nervous system, among others.” The term nutraceutical was first used in 1989 by Dr. Stephen DeFelice, from the United States Foundation for Innovation in Medicine.

One embodiment of the present invention is to select active ingredients capable of producing anti-depressant, anti-stress and anti-anxiety effects based on the biological activity, and physical and chemical properties of the active ingredients of the nutraceutical liquid formulation of the invention.

Manufacturing Process

The manufacturing process of a nutraceutical formulation in liquid form for the treatment of DSA comprises the following steps:

1. Full or whole seeds are selected according to its inner solid content and uniform yellowish appearance.

2. Seeds are cleaned so as to remove scales, black seeds, dust particles and small pieces of stem.

3. Seeds are washed with water for a suitable period of time with a jet of water. Water must be treated by any means known in the art in order to remove any dust particles, chlorine, for example, through filters comprising cellulose and activated carbon cartridges, and finally an ultraviolet treatment to eliminate microorganisms.

4. Seeds are placed on a container and pretreated water is added in an amount of 10 to 15 parts by volume and one part by weight of seeds. Seeds are soaked for 8 to 16 hours at a temperature of 0-10° C.

5. Seeds are washed with water for 8 to 12 minutes with a jet of pretreated water.

6. Soaked and cleaned seeds are ground, with pretreated water, the process is carried out at a temperature of between 0-10° C., in a proportion of 5 to 10 parts by volume and one part by weight of seeds. The grinding equipment can be selected, for example, from industrial blenders or grinding means having a knife system, among other commercial grinding equipment. The ground seeds are allowed to settle for a period of time between 10 and 15 minutes, at a temperature of 10° C. to 30° C.

7. The suspension is filtered in order to separate the amaranth beverage from the insoluble components and recover soluble proteins in aqueous solution, lipids formed in the suspension and soluble carbohydrates, thereby removing non-soluble carbohydrates such as starches, some lipids not forming part of the suspension, non-soluble proteins in aqueous solution, and fiber. The filtering process can be performed by using any low-scale or industrial filtering system, for example, but not limited to, a fine-woven flannel fabric, or using an industrial filtering equipment or another kind of industrial filter for slurry.

8. The suspension is agitated to homogenize its content for 10-20 minutes using a low-scale or industrial homogenization system.

9. The amount of protein in the amaranth beverage is determined by using procedures known in the art, for example: bicinchoninic acid assay, Biuret reaction, Lowry method, absorption in the ultraviolet region by using a spectrophotometer or modern equipment (Nanodrop), and immunologic methods, preferably the bicinchoninic acid assay is used.

10. The amount of fat in the amaranth beverage is determined using known methods such as the Gerber, Babcock, Tesa technique, Rosse-Gottlier and Mojonnier methods, preferably the Gerber method is used.

11. The amount of carbohydrates present in the beverage is determined, using known methods such as the Fehling method, or by weight differences of the dry matter and the amount of protein, fat and ashes, and the weight of water lost during the drying step, the Fehling method is preferred.

12. The amount of minerals in the amaranth beverage is determined by methods such as flame atomic absorption spectrophotometry, hydride generation techniques and graphite oven; or in a digestion microwave oven with pressure and temperature control, preferably, the flame atomic absorption spectrophotometry method is used.

13. The amount of vitamins in the amaranth beverage is determined by using thermal, spectrophotometric and fluorometric assays, and by high-performance liquid chromatography (HPLC). Preferably the reverse-phase high-performance liquid chromatography (HPLC) method is used.

14. An oxidant of the group comprising ascorbic acid, ascorbic acid salts, erythorbic acid, erythorbic acid salts, sodium sulfite, potassium sulfite, dibasic sodium sulfite, dibasic potassium sulfite, sodium hyposulfite, potassium hyposulfite, potassium pyrosulfite, sodium citrate, potassium citrate and L-cysteine hydrochloride. Ascorbic acid is used, and the amount of antioxidant ranges from 0.01 to 1.00 parts by weight per 100 parts by dry weight of whole seeds.

15. A stabilizer selected from the group comprising agar-agar, acacia (gum Arabic), ammonium alginate, calcium alginate, carrageenin, potassium alginate, sodium alginate, gum Ghatti, gum Sterculia (gum Karaya), tragacanth (gum tragacanth), Guar gum, sodium hydrogen carbonate, trisodium citrate, calcium chloride and disodium phosphate is added. For example, carrageenin can be employed, and the amount of stabilizer ranges from 0.005 to 0.03 parts by weight per 100 parts by dry weight of seed, preferably from 0.01 to 0.015.

16. A natural flavor is added during the grinding process of the soaked seeds, said flavoring can be cinnamon.

17. Optionally, other natural flavors from fruits and seeds can be added, which are selected from the group comprising, but not limited to: guava, apple, pear, mango, soursop, banana, peach, papaya, quince, strawberry, walnut, passion fruit, cherry, raspberry, blackberry, blueberry, grape, apricot, among others or combinations thereof.

18. Optionally, a food supplement from a group comprising vitamin C, vitamin D, vitamin E, or vitamin B complex can be added to the amaranth beverage.

19. Optionally, a colorant selected from a group comprising white, red, and yellow, among others can be added to the amaranth beverage.

20. The suspension is agitated to make it homogeneous, and 1 N sodium hydroxide is added until a pH 11.0 is reached, and agitation continues for 2 hours until a complete homogenization of nutrients is obtained at this pH value.

21. The suspension is decanted to obtain a first nutrient-rich solution for the nutraceutical formulation; the remaining solution and starch are filtered in a process using industrial filter press equipment or another type of industrial slurry filter. The liquid material obtained from filtration is incorporated to the liquid separated by decantation. In a second extraction, the solid material is resuspended in a 10% NaCl solution and continually agitated for 4 hours. Both solutions obtained in the first and second steps are mixed together and agitated for 1 hour in order to obtain an ideal mixture of nutrients.

22. The obtained liquid undergoes an isoelectric precipitation process at pH between 6.5 and 7.5, preferable a pH 7.0. The isolates prepared contain the nutraceutical formulation nutrients of the present invention.

23. According to the described process, a nutraceutical formulation is obtained, characterized in that it contains the following nutritional elements: 43 to 47% protein; 1 to 3% lipids; 1 to 2% salts; 39-43% carbohydrates; 3.0-3.50 soluble fiber and 3.5 to 4.0% moisture.

24. The nutraceutical formulation contains an average of 43 to 47% superior quality protein, with a high content of essential amino acids constituting about 48 to 51%, and preferably 49.3% of its composition. The amino acids composition of the most abundant globulin contained in the amaranth beverage is: 4.5 to 4.7% alanine, 2.4 to 3.5% arginine, 9.2 to 11.0% asparagine, 5.2 to 5.6% aspartic acid, 1.4 to 1.8% cysteine, 4.4 to 5.1% glutamine, 4.4 to 4.8% glutamic acid, 5.0 to 5.6% glycine, 2.2 to 2.5% histidine, 6.3 to 6.8% isoleucine, 8.4 to 8.7% leucine, 6.7 to 7.0% lysine, 2.2 to 2.6% methionine, 5.3 to 5.8% phenylalanine, 3.5 to 3.8% proline, 6.7 to 7.0% serine, 5.7 to 6.1% threonine, 2.1 to 2.5% tryptophan, 4.7 to 5.0% tyrosine and 6.1 to 6.4% valine. The amaranth beverage contains a high amount of the essential amino acids lysine, tryptophan, methionine and cysteine. These amino acids are essential to health, resulting in a beverage that satisfactory fulfills the requirements recommended by the Food and Agricultural Organization (FAO), for an optimal human nutrition.

Table 1 shows the composition of the powdered nutraceutical formulation (per 100 grams).

| | Powdered Nutraceutical | |
|---|---|---|
| Analysis | Formulation (100 g) | |
| Energy content | 358.55 (Kcal/100 g)^{a} | |
| | 1,523.40 (KJ/100 g)^{b} | |
| Proteins* | 44.50 | g |
| Fat (Lipids) | 1.75 | g |
| of which saturated fat | 0.50 | g |
| Total carbohydrates | 41.20 | g |
| of which sugars | 4.00 | g |
| Dietary fiber | 3.25 | g |
| Ashes | 1.60 | g |
| Cholesterol | 0.00 | g |
| Sodium, mg/100 g | 22.99 | mg |
| Potassium, mg/100 g | 411.00 | mg |
| Calcium, mg/100 g | 298.00 | mg |
| Phosphorous, mg/100 g | 862.00 | mg |
| Manganese, mg/100 g | 4.00 | mg |
| Iron, mg/100 g | 0.76 | mg |
| Thiamin (Vitamin B1) | 0.14 | mg |
| Riboflavin (Vitamin B2) | 0.34 | mg |
| Vitamin E (equivalent to tocopherol) | 10.00 | mg |
| Ascorbic acid (Vitamin C) | 4.50 | mg |
| Moisture | 3.70 | g |

*Factor of 6.25
^{a}energy or energy value in calories/100 g of material = 4 (% nitrogen free extract + total protein) + 9 (ether extract).
^{b}energy or energy value in Joule/100 g of material = 17 (% nitrogen free extract + total protein) + 38 (ether extract).

According to Mexican Standard NOM-008-SCFI-1993. The General System of Units of Measurement, states that “the decimal separator must be a comma (,). If the magnitude of the number is less than the unit, the decimal separator must be preceded by a zero”. Therefore, the results in this document must be interpreted in this way.

Secretaría de Salubridad y Asistencia. Dirección General de Investigación en Salud Pública. Técnicas para el análisis fisicoquímico de alimentos. Year 1995.

NOM-116-SSA1-1004; NMX-F-089-S-1978; NMX-F-608-NORMEX-2002 and NOM-051-SCFI-SSA1-2010.

25. It is worth mentioning that in the steps 14 and 15 where an antioxidant and a stabilizer are added, the purpose is to avoid precipitation of the protein and carbohydrates, so as to favor the homogenous appearance and extend the storage span or shelf life, thus addressing the technical problem present in similar products and processes of the prior art.

26. Another embodiment of the invention is a soluble powdered nutraceutical formulation, which suspension obtained is allowed to settle for 2 to 4 hours and then undergoes a drying process, which can be performed in, for example, but not limited to, a spray dryer, or a laminar flow drying oven, in order to obtain a slightly yellowish powder.

27. In another embodiment of the invention, different vitamins from the group comprising vitamin C, vitamin D, vitamin E or vitamin B complex, among other, can be added to the soluble powdered nutraceutical formulation.

28. In another embodiment of the invention, the soluble powdered nutraceutical formulation can contain different preservatives from a group comprising ascorbic acid, ascorbate salts, potassium sorbate, propionic acid, sodium and potassium tartrate, sodium citrate, succinic acid, tocopherols, sorbic acid, and sodium sesquicarbonate.

29. In an optional embodiment of the invention, colorants from the group comprising white, red and yellow including different coloring intensities thereof, can be added to the soluble powdered nutraceutical formulation

By way of illustration of the present invention, the following examples are presented showing various processes for producing compounds according to the invention, then the use of the same and advantages thereof, without limiting the scope of the invention.

### EXAMPLE 1

50 to 250 grams of whole or full seeds are selected according to its inner solid content, by breaking the seed and inspecting them visually. Seeds are cleaned so as to remove black seeds, small pieces of stem, seed scales and dust particles. Seeds are washed with microorganism-free water. Seeds are soaked for various hours at a temperature between 4 and 36° C. using microorganism-free water. The soaked seeds are ground, in a hammer mill grinder until a suitable suspension is obtained. The suspension is filtered using a filter press in order to remove insoluble solids. The composition of insoluble solids is: fiber, protein that is non-soluble in aqueous solution, a portion of lipids and carbohydrates.

The protein is extracted using the alkali extraction and isoelectric methodology. The pH of the solution is increased to pH 11.0, using 0.1 N NaOH and continuous agitation for 2 hours for extraction, the suspension is filtered using industrial filter press equipment or another kind of slurry filter. In a second extraction, the solid material is resuspended in a 10% NaCl solution and continually agitated for 4 hours. The suspension is allowed to settle for one hour and then decanted. All solutions obtained in the first and second steps are mixed together and agitated for 1 hour in order to obtain an ideal mixture of nutrients. The liquid obtained undergoes an isoelectric precipitation process at pH of 7.5, using 0.1 N HCl. The obtained isolates contain the nutraceutical formulation nutrients of the present invention. The suspension obtained is allowed to settle for 2 to 4 hours, and then undergoes a drying process, which can be performed in, for example, but not limited to, a spray dryer in order to obtain a slightly yellowish powder.

The nutraceutical formulation composition is as follows: 43 to 47% protein; 1 to 3% lipids; 1 to 2% salts; 39-43% carbohydrates; 3.0-3.50 soluble fiber, and 3.5 to 4.0% moisture.

### EXAMPLE 2

50 to 250 grams of whole or full seeds are selected according to its inner solid content, by breaking the seed and inspecting them visually. Seeds are cleaned so as to remove black seeds, small pieces of stem, seed scales and dust particles. Seeds are washed with microorganism-free water. Seeds are soaked for various hours at room temperature using microorganism-free water. The soaked seeds are ground, in presence of cinnamon as a natural flavoring agent in a hammer mill grinder, and a stabilizer is added, until a suitable suspension is obtained. The suspension is filtered using a filter press in order to remove insoluble solids. The protein is extracted using the alkali extraction and isoelectric methodology. The pH of the solution is increased to pH 11.0, using 0.2 N NaOH and continuous agitation for 2 hours for extraction, the solution is decanted to obtain a first nutrient-rich solution for the nutraceutical formulation, the remaining solution and starch are filtered in a process using industrial filter press equipment or another type of industrial slurry filter. The liquid material thus obtained from the filtration step is incorporated to the liquid separated by decantation. In a second extraction, the solid matter is resuspended in a 20% NaCl solution and continually agitated for 4 hours. The suspension is allowed to settle for two hours and then decanted. Both solutions obtained in the first and second steps are mixed together and agitated for 1 hour in order to obtain an ideal mixture of nutrients. The pH of the liquid thus obtained is adjusted to pH 7.0 using 0.1 N HCl. The isolates prepared contain the nutraceutical formulation nutrients of the present invention. The suspension obtained is allowed to settle for 2 to 4 hours, and then undergoes a drying process, which can be performed in, for example, but not limited to, a laminar flow drying oven in order to obtain a slightly yellowish powder.

The nutraceutical formulation composition is as follows: 43 to 47% protein; 1 to 3% lipids; 1 to 2% salts; 39-43% carbohydrates; 3.0-3.50 soluble fiber, and 3.5 to 4.0% moisture.

### EXAMPLE 3

50 to 250 grams of whole or full seeds are selected according to its inner solid content, by breaking the seed and inspecting them visually. The black seeds, which correspond to wild amaranth, are not removed, but the small pieces of stem, seed scales and dust particles are removed. In this procedure wild seeds are used, which contain similar amounts of protein, lipids, carbohydrates and water as the cultivated seed. Seeds are washed with microorganism-free water. Seeds are soaked for a short period of time at room temperature using microorganism-free water. The soaked seeds are ground, in presence of cinnamon as a natural flavoring agent, in a knife mill grinder, and a stabilizer agent is added, until a suitable suspension is obtained. The suspension is filtered using a filter press or fabric filter fitted with a vacuum system in order to carry out the process in a fast and efficiently so as to remove insoluble solids.

The nutraceutical formulation is extracted using semipermeable membranes and pilot- or industrial-scale ultrafiltration equipment for the separation of components and, particularly, for the concentration and purification of proteins.

The isolates prepared contain the nutraceutical formulation nutrients of the present invention. The suspension obtained is allowed to settle for 2 to 4 hours and then undergoes a drying process, which can be performed in, for example, but not limited to, a spray dryer or a laminar flow drying oven in order to obtain a slightly yellowish powder.

The nutraceutical formulation composition is as follows: 43 to 47% protein; 1 to 3% lipids; 1 to 2% salts; 39-43% carbohydrates; 3.0-3.50 soluble fiber, and 3.5 to 4.0% moisture.

### EXAMPLE 4

50 to 250 grams of whole or full seeds are selected according to its inner solid content, by breaking the seed and inspecting them visually. Seeds are cleaned so as to remove black seeds, small pieces of stem, seed scales and dust particles. Seeds are washed with microorganism-free water. Seeds are soaked for a short period of time at room temperature using microorganism-free water. The soaked seeds are ground, in presence of cinnamon as a natural flavoring agent, in a hammer mill grinder and a stabilizer is added, until a suitable suspension is obtained. The suspension is filtered using a filter press or a filter equipped with a screening device made of stainless steel, in order to remove insoluble solids.

The protein is extracted using the dialysis or ultrafiltration process to concentrate and purify proteins. Said processes are based on the use of semipermeable membranes which allow small molecules to pass through them but not proteins or another macromolecules.

The nutraceutical formulation composition is as follows: 43 to 47% protein; 1 to 3% lipids; 1 to 2% salts; 39-43% carbohydrates; 3.0-3.50 soluble fiber, and 3.5 to 4.0% moisture.

### EXAMPLE 5

In the efficacy measurement, a successful trial of antidepressants, anti-stress and anti-anxiety drugs requires that at least that 50% of the patients are responsive to the medication, which means that the depression, stress, anxiety, schizophrenia and bipolar symptoms have been reduced at least by 50%.

A group of 10 individuals with depression, stress and anxiety were administered six capsules (two capsule per meal, i.e. breakfast, lunch and dinner), each containing 400 g of the nutraceutical formulation, the test was successful because all 10 individuals showed a significant change in mood, by reducing by 90 to 95%, in less than half hour, the depression, stress, anxiety, schizophrenia and bipolar symptoms.

### EXAMPLE 6

A group of 10 individuals with schizophrenia, bipolarity and extreme depression, were administered six capsules (two capsules per meal, i.e. breakfast, lunch and dinner), each containing 400 g of the nutraceutical formulation, the trial was successful because all 10 individuals showed a significant change in mood, by reducing by 90 to 95%, in less than half hour, the extreme symptoms of schizophrenia, bipolar disorder, depression, stress and anxiety.

The object of the invention is related to address problems of great relevance in the priority field of public health, which significantly affects people in general, that is to say, depression, stress, anxiety (DSA), schizophrenia and bipolar disorder.

The nutraceutical formulation composition is as follows: 43 to 47% protein; 1 to 3% lipids; 1 to 2% salts; 39-43% carbohydrates; 3.0-3.50 soluble fiber, and 3.5 to 4.0% moisture.

The above examples have been provided only for the purpose of exemplification and are not intended to limit the scope or contents of the invention. The invention is described in further detailed with reference to the following claims.

1-30. (canceled)

31. A nutraceutical formulation of amaranth wherein Amaranthus hypochondriacus contains proteins with a high content of essential amino acids, unsaturated fatty acids, squalene, antioxidants, minerals and carbohydrates.

32. A nutraceutical formulation of amaranth in accordance with claim 31 in the form a soluble powder and/or capsules.

33. A nutraceutical formulation of amaranth in accordance with claim 31, wherein the formulation is a liquid.

34. A nutraceutical formulation of amaranth in accordance with claim 31, wherein the formulation is characterized by a solid presentation content of 43 to 47% of protein by weight of the total composition, 1-3% of lipids by weight of the total composition, 1-2% of salts by weight of the total composition, from 39 to 43% of carbohydrates by weight of the total composition, 3.0 to 3.50% of soluble fiber by weight of the total composition and from 3.5 to 4.0% moisture by weight of the total composition

35. A nutraceutical formulation of amaranth in accordance with claim 31, further characterized as having a good solubility in water at different temperatures from 4 to 60° C.

36. A process of preparing a nutraceutical product of Amaranth which includes selecting, clean seeds by sieves, washing the seeds with water, soaking the seeds with water free of microorganisms, washing the seeds soaked with water free of microorganisms, grinding the seeds, homogenizing the suspension of ground seeds, decanting or filtering the suspension, performing an alkaline extraction and isoelectric precipitation, wherein the extraction of the nutraceutical formulation is through a process of ultrafiltration and dialysis, subsequently adding an antioxidant, adding a stabilizer, adding a nutritional supplement, adding a colorant, adjusting the volume of the solution to a desired concentration of the nutraceutical formulation, homogenizing the suspension through agitation and drying the nutraceutical formulation.

37. The process in accordance with claim 36, also characterized by including an artificial or natural flavoring and coloring to the nutritional supplement.

38. The process in accordance with claim 36, also characterized by including a step of determining the amount of protein, amount of fat, and quantity of carbohydrates in the solid presentation.

39. The process in accordance with claim 36, also characterized by including a step of drying the liquid product to produce a solid product, in the form of soluble powder.

40. The process in accordance with claim 39, characterized by including a step of adding a flavoring to the solid product.

41. The process in accordance with claim 36, characterized by including a step wherein the seed selection is by visual inspection, in which, the seed must have a solid internal content and a uniform appearance of yellow, no Earth particles, nor seeds films and even small pieces of stem.

42. The process in accordance with claim 36, characterized by including a step of washing the seeds using water that has been treated by various filters that contain cellulose cartridges to remove dust particles and activated carbon cartridges to remove chlorine, and is finally treated with UV light to eliminate microorganisms.

43. The process in accordance with claim 36, characterized by including a step of soaking the seeds with treated water for 8 to 16 hours, at a temperature of 0° to 10° C.

44. The process in accordance with claim 42, characterized by including a step of washing the soaked seeds for 8 to 12 minutes in a stream of previously treated water, at a temperature between 0 and 10° C. and between 5 to 10 parts by volume per one part of seed weight.

45. The process in accordance with the claim 36, characterized by including a step of grinding the seeds to get a suspension by use of an industrial Blender, hammer mill or grinding media which have a system of blades.

46. The process in accordance with claim 36, characterized by a step of adding a flavor which is natural cinnamon in an amount which can range from 1.0 to 3.5 parts by weight per 100 parts by dry weight of the whole seed.

47. The process in accordance with claim 45, characterized by including a step of milling time which varies between 5 to 9 minutes.

48. The process in accordance with claim 45, characterized by including a step to set aside the suspension for a period of time between 6 and 15 minutes at a temperature of 10° C. to 30° C., and after that subjecting it to a stage of homogenization through agitation of the suspension for 10 to 20 minutes through a system of small-scale or industrial homogenization.

49. The process in accordance with the claim 45, characterized by including a step of filtration of the suspension through the use of a fabric of flannel, tissue usually carded, napping and slightly felting wool and the effect of gravity either using press industrial filtration equipment or using decanting processes filtration through semipermeable membranes, ultrafiltration membranes, dialysis scale plant pilot or industrial or other type of filtration of suspensions for industrial use.

50. The process in accordance with claim 36 characterized by including a step of adding of an antioxidant agent in the limits of 0.01 to 1.0 parts in dry seed weight, selected from a group consisting of ascorbic acid, salts of the ascorbic acid, erythorbic acid, potassium sulfite sodium sulfite dibasic, potassium sulfite dibasic, sodium hyposulfite and sodium citrate, potassium citrate and L-cysteine hydrochloride, preferably ascorbic acid.

51. The process in accordance with claim 36, characterized by including a step of using a stabilizing agent that is added to the suspension between 0.005 to 0.03 parts by weight per 100 parts by weight of dry seed, selected from a group consisting of agar-agar, acacia (Arabic gum), carrageenan, sodium alginate, Ghatti gum, Esterculia gum (Karaya gum), guar gum, calcium chloride and disodium phosphate, preferably carrageenan.

52. The process in accordance with claim 37, characterized because it including a step of selecting an artificial flavoring from the group that includes chocolate, vanilla, nuts, banana, coconut or mixtures thereof.

53. The process in accordance with claim 37, characterized by includes a step of adding the natural flavoring which is selected from the group consisting of guava, Apple, mango, peach, mamey, papaya, walnut, passion fruit, cherry, raspberry, BlackBerry, cranberry, grape or mixtures thereof.

54. The process in accordance with claim 37, characterized by including a step of adding a nutritional supplement which is selected from a group comprising vitamin C, vitamin D, vitamin E or B complex.

55. The process in accordance with claim 37, characterized by including a step to add coloring which is selected group comprising white 1, red 40 and yellow 5 or mixtures thereof.

56. The process in accordance with claim 36, characterized by including a step of selecting the concentration of the nutraceutical formulation to adjust the volume of the solution to 43 up to 47% protein by weight of the total composition, preferably 44.5% by weight of the total composition.

57. Use of a nutraceutical product from Amaranthus hypochondriacus, for the treatment of disorders of the human behavior such as depression, stress, anxiety, in its extreme cases such as, schizophrenia and bipolar disorder.

58. Use in accordance with claim 57, wherein early or preliminary stages of such human behavior disorders are selected from disorders such as irritable mood or under, loss of pleasure in usual and sexual activities, difficulty sleeping or sleep too much, change in appetite with gain or loss of weight, fatigue and lack of energy feelings of uselessness, lack of self-love and guilt, difficulty concentrating, slow or fast movements, feelings of sadness of pathologic type and abandonment, repetitive thoughts of death or suicide to its consummation and distractions frequent, crisis of anguish and anxiety, low working efficiency, delusions, hallucinations, affectivity flat, speech and abrupt changes in mood.

59. The use of the nutraceutical formulation of amaranth in accordance with claim 57, wherein it can be ingested by healthy individuals.

60. A nutraceutical delivery vehicle acceptable for a formulation of amaranth in capsule form.

61. The process in accordance with claim 40, characterized by including a step of adding a flavor which is natural cinnamon in an amount in a range from 1.0 to 3.5 parts by weight per 100 parts by dry weight of the whole seed.

62. The process in accordance with claim 48, characterized by including a step of filtration of the suspension through the use of a fabric of flannel, tissue usually carded, napping and slightly felting wool and the effect of gravity either using press industrial filtration equipment or using decanting processes filtration through semipermeable membranes, ultrafiltration membranes, dialysis scale plant pilot or industrial or other type of filtration of suspensions for industrial use.

63. The process in accordance with claim 37, characterized by includes a step of adding the natural flavoring which is selected from the group consisting of guava, Apple, mango, peach, mamey, papaya, walnut, passion fruit, cherry, raspberry, BlackBerry, cranberry, grape or mixtures thereof.

64. The process in accordance with claim 45, characterized by including a step of using a stabilizing agent that is added to the suspension between 0.005 to 0.03 parts by weight per 100 parts by weight of dry seed, selected from a group consisting of agar-agar, acacia (Arabic gum), carrageenan, sodium alginate, Ghatti gum, Esterculia gum (Karaya gum), guar gum, calcium chloride and disodium phosphate, preferably carrageenan.
